(19) United States
(12) Patent Application Publication
(10) Pub. No.: US 2014/0057851 A1
(43) Pub. Date: Feb. 27, 2014

(54) Anti-Angiogenic Gene Therapy With Soluble VEGF Receptors -1, -2 and -3 Together With Paclitaxel Prolongs Survival Of Mice With Human Ovarian Carcinoma

(71) Applicant: Seppo YLA-HERTTUALA, Kuopio (FI)

(72) Inventor: Seppo YLA-HERTTUALA, Kuopio (FI)

(73) Assignee: WKD Holding Oy, Kuopio (FI)

(21) Appl. No.: 13/969,763

(22) Filed: Aug. 19, 2013

Related U.S. Application Data
(60) Provisional application No. 61/692,828, filed on Aug. 24, 2012.

(51) Int. Cl. A61K 38/17; A61K 31/337

(52) U.S. Cl. CPC A61K 38/179; A61K 31/337
USPC 514/19.3; 514/44 R

(57) ABSTRACT

Anti-angiogenic gene therapy with a combination of soluble Vascular Endothelial Growth Factors (sVEGFR) improves the efficacy of chemotherapy with paclitaxel for reducing ovarian cancer mean tumor volume (in cubic millimetres) as measured using magnetic resonance imaging. The study groups were: AdLacZ control, combination of AdsVEGFR-1, -2 and -3, combination of AdsVEGFR-1, -2, -3 and paclitaxel, bevacizumab monotherapy, paclitaxel monotherapy and carboplatin monotherapy. Effectiveness was assessed by survival time and surrogate measures such as sequential MRI, immunohistochemistry, microvessel density and tumor growth. Antiangiogenic gene therapy combined with paclitaxel significantly prolonged the mean survival compared to the controls and all other treatment groups (p=0.001). Tumors of the mice treated by gene therapy were significantly smaller than in the control group (p=0.021). The mean vascular density and total vascular area were also significantly smaller in the tumors of the gene therapy group (p=0.01).

### RELATED APPLICATIONS

This application claims priority from U.S. provisional filing Ser. No. 61/692,828, filed 24 Aug. 2012.

### GOVERNMENT OWNERSHIP INTEREST

None.

### BRIEF DESCRIPTION

Ovarian cancer is the leading cause of mortality from gynaecologic cancers.¹ By the time of diagnosis nearly 70% of the patients with ovarian carcinoma have widely disseminated disease with intraperitoneal carcinosis and ascites. Prognosis of these patients remains poor and 5-year survival is 30%, despite of the optimal cytoreductive surgery and combination chemotherapy. Platinum compounds, at present mainly carboplatin, have remained the single most active drugs in the treatment of ovarian carcinoma. As the first line therapy, platinum is combined with taxanes (paclitaxel) to avoid platinum resistency, which is a major problem in the chemotherapy.² To improve the treatment effect new antiangiogenic treatments have been under investigation during the last few years also in ovarian cancer.

Angiogenesis plays a vital role in tumor growth and metastasis. Cancer cells need neovascularisation to grow and invade.³ VEGF is overexpressed in ovarian carcinoma cells and associated with poor prognosis.^{4,5} VEGF family members (VEGF-A, -B, -C, -D and placental growth factor) mediate their effects through VEGF receptors VEGFR-1, -2 and -3, known also as Flt-1, KDR and Flt-4, respectively.⁶ VEGFR-2, which binds VEGF-A, -C and -D, is considered to play a major role in angiogenesis, vasculogenesis and vascular permeability, whereas VEGFR-1 (binding VEGF-A, -B and placental growth factor) stimulates tumor growth and metastasis by angiogenesis.⁷ VEGFR-3 binds VEGF-C and -D and is essential for tumor dissemination via lymphatic vessels.^{6,8,9} Monoclonal anti-VEGF-antibody, bevacizumab, has been used as a combination and consolidation therapy in phase III studies.¹⁰ Preliminary results have shown efficacy of the treatment in terms of prolonged progression free survival, but effect on overall survival needs to be waited.

Several phase I studies of gene therapy for the ovarian carcinoma have been published, many of them with adenoviral vectors restoring tumor suppressor genes like p53 and BRCA-1, inhibiting oncogenes, such as erB2 and EGFR or using suicide genes. These strategies have shown only limited efficacy and no clinical applications have yet been established.¹¹ There thus remains an unmet need for safe and effective therapy for ovarian cancer.

To further expand our earlier results on sVEGFR gene therapyl² and provide a solution for this unmet need, we compared the effects of this therapy to traditional chemotherapy and to the treatment with monoclonal anti-VEGF antibody in an ovarian cancer xenograft model that closely resembles human ovarian cancer and shows a very aggressive behaviour.¹³ Gene therapy included a combination of human sVEGFR-1, -2 and -3. sVEGFRs used in this study lack transmembrane and intracellular tyrosine kinase domains and do not initiate signal transduction.⁶ All constructs contain an immunoglobulin Fc domain to ensure efficient dimerization of the soluble decoy receptors.

In addition, the combined effect of gene therapy and chemotherapy was studied by adding paclitaxel to the sVEGFR treatment. MRI was applied for the timing of gene therapy to treat sizable tumors, instead of a micrometastatic disease, and to follow tumor progression in vivo.¹³ To our knowledge this is the first study to combine antiangiogenic gene therapy with chemotherapy in ovarian cancer and to compare the efficacy of antiangiogenic gene therapy to the current treatment modalities including monoclonal anti-VEGF-antibody therapies. Results are promising and support clinical testing of the concept.

### BRIEF DESCRIPTION OF THE DRAWINGS

FIG. 1. Protocol of the study. Tumors developed within 3 weeks after the inoculation of the tumor cells. The presence of all tumors was verified by MRI before starting the therapy. Tumors were observed weekly until the death of the mice. Plasma samples were collected 6, 13, and 27 days after the treatment. MRI, magnetic resonance imaging.

FIG. 2. MRI measurements of tumor growth and tumor weights. (a) At the time of the 2^{nd} MRI (1 week after the treatment) the mean tumor volume of the gene therapy group and the combined therapy group was smaller than tumor volumes in control, bevacizumab and single paclitaxel groups (p=0.001). In the 3^{rd} MRI (2 weeks after the treatment) the mean tumor volume of the combined gene therapy with paclitaxel group was significantly smaller than in all other treatment groups (p=0.002). Mean tumor volume of the gene therapy alone was also smaller than in control, bevacizumab and paclitaxel groups (p=0.004; p=0.036 respectively). (b) The mean tumor weights at the end of the follow-up. Gene therapy group had smaller tumors than the control (p=0.02) and paclitaxel group. Tumors of combined gene therapy and paclitaxel group were also smaller than in controls and single paclitaxel group (p=0.037, p=0.048). (c) MRI images of the growth of the ovarian tumors in control (LacZ), gene therapy (sR-1, -2, -3) and bevacizumab treated mouse before (MRI I), 1 week (MRI II) and 2 weeks (MRI III) after the treatment. Tumors are marked with arrows and circled. MRI, magnetic resonance imaging. *P<0.05; **P<0.001; ***P<0.001.

FIG. 3. Histology and measurements of the intraperitoneal ovarian tumors. (a) Hematoxylin-eosin staining of serous cystadenocarcinoma in control group (AdLacZ). Focal necrosis and connective tissue were present in the tumor tissue in gene therapy group (sVEGFR-1, -2, -3). (b) CD-34 positive microvessels in tumor tissues of different treatment groups. (c) Microvessel density (MVD, microvessels/mm²) was significantly reduced in mice treated with gene therapy alone (p=0.001) and together with paclitaxel (p=0.008). Gene therapy significantly reduced the total area of tumors covered by microvessels (p=0.0005) (TVA, tumor vascular area). *P<0.05; **P<0.001 ; ***P<0.001. Magnification, ×100; Bar, 100 μm. Error bars, SEM.

FIG. 4. Kaplan Meier survival analysis curves. The mean survival was significantly prolonged by combination of gene therapy and paclitaxel (p=0.001). Overall, prolongation of the survival was also observed by gene therapy compared to bevacizumab (p=0.05).

### DETAILED DESCRIPTION

We compared effects of antiangiogenic gene therapy with a combination of soluble sVEGFR-1, sVEGFR-2 and sVEGFR-3 to chemotherapy with carboplatin and paclitaxel, and to antiangiogenic monoclonal anti-VEGF-antibody bevacizumab in an intraperitoneal ovarian cancer xenograft model in mice (n=80). Gene therapy was also combined with chemotherapy. Therapy was initiated when sizable tumors were confirmed in magnetic resonance imaging (MRI). Adenovirus-mediated gene transfer was performed intravenously (2×10⁹ pfu); while chemotherapy and monoclonal anti-VEGF-antibody were dosed intraperitoneally. The study groups were: AdLacZ control (n=21); combination of AdsVEGFR-1, -2 and -3 (n=21); combination of AdsVEGFR-1, -2, -3 and paclitaxel (n=9); bevacizumab (n=14); paclitaxel (n=9); and carboplatin (n=5). Effectiveness was assessed by survival time and surrogate measures such as sequential MRI, immunohistochemistry, microvessel density and tumor growth. Antiangiogenic gene therapy combined with paclitaxel significantly prolonged the mean survival of mice (25d) compared to the controls (15d) and all other treatment groups (p=0.001). Bevacizumab treatment did not have any significant effect on the survival. Tumors of the mice treated by gene therapy were significantly smaller than in the control group (p=0.021). The mean vascular density and total vascular area were also significantly smaller in the tumors of the gene therapy group (p=0.01). These results show potential of the antiangiogenic gene therapy to improve efficacy of chemotherapy with paclitaxel and support testing of this approach in a phase I clinical trial for the treatment of ovarian cancer.

### Materials and Methods

### Cell Line

SKOV-3m cell line has been characterized earlier.¹³ Cells were cultured in McCoy's 5A medium (Gibco, Invitrogen, Life technologies). Cancer cells were tested to be mycoplasma free. Before in vivo inoculation the cells were trypsinized and counted.

### Chemotherapy and Anti-VEGF-Antibody

Carboplatin 10 mg/ml infusion concentrate was used at a dose of 80 mg/kg for the primary treatment of Balb/cA-nu mice. The dose per Balb/cA-nu was 1.6 mg/500 μl NaCl 0.9%.¹⁴ Paclitaxel 6 mg/ml concentrate was used at a dose of 20 mg/kg. The dose per nude mouse was 320 μg/500 μl NaCl 0.9%.¹⁵ Bevacizumab 25 mg/ml concentrate was used at a dose of 5 mg/kg and the dose per nude mouse was 100 μg/500 μl NaCl 0.9%.¹⁶ It was injected every fifth day.

### Viral Vectors

Adenoviral vectors encoding sVEGFR-1-IgG fusion protein^{17,18,19}, sVEGFR-2-IgG fusion protein, sVEGFR-3-IgG fusion protein^{20,21} and LacZ (AdLacZ) as a control vector were used for the study. Replication-deficient E1-E3 deleted clinical GMP-grade adenoviruses were produced in 293 cells. Adenoviruses were analyzed to be free from helper viruses, lipopolysaccharides and bacteriological contaminants.^{22,23}

### Animal Model

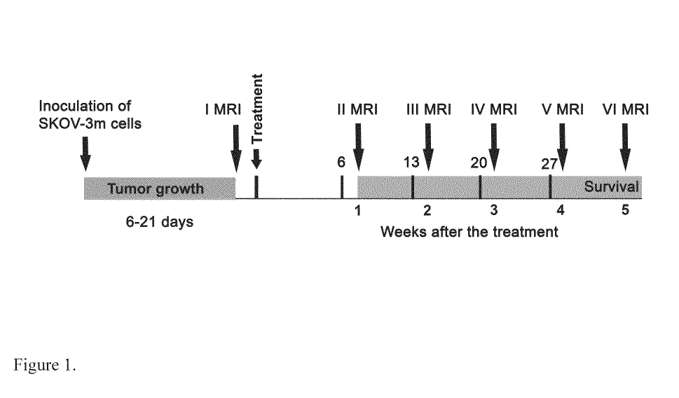

Eight to ten-weeks-old (n=80) Balb/cA-nu female nude mice were used for the studies. Ovarian carcinoma was produced by inoculating 1×10⁷ SKOV-3m cells into the peritoneal cavity of nude mice with a 22 G needle.¹³ Development of the ovarian cancerous tumors was followed by sequential MRI. When the first solid, measurable tumor was detected in MRI, gene transfer or other treatment was started on the following day.¹² Mice were randomly divided into six groups: 21 animals received combination of AdsVEGFR-1, AdsVEGFR-2 and AdsVEGFR-3 (2×10⁹ pfu/200 μl) once; 14 animals received bevacizumab 100 μg/500 μl every fifth day until sacrifice; nine animals received paclitaxel 320 μg/500 μl once as a single therapy and five animals received carboplatin 1.6 mg/500 μl once as a single treatment; nine animals received a combination of AdsVEGFR-1, AdsVEGFR-2 and AdsVEGFR-3 (2×10⁹ pfu/200 μl) once, and after one week paclitaxel 320 μg/500 μl as a one shot; 21 control animals received AdLacZ (2×10⁹ pfu) (Table 1, FIG. 1). Gene transfer was performed intravenously (i.v.) via tail vein in the final volume of 200 μl in 0.9% saline. Paclitaxel, bevacizumab and carboplatin were dosed intraperitoneally (i.p.).

MRI was done weekly after gene transfer or treatment and tumor volumes were measured. The overall follow-up time lasted until the appearance of significant symptoms necessitating sacrifice or to the death. At the time of death, all tumor tissue, liver, spleen, kidneys and lungs were harvested and tumor masses were weighed. Ascites fluid was collected in a syringe. The mice were kept in a pathogen free isolated unit at the National Experimental Animal Center of the University of Eastern Finland. Food, water and sawdust bedding were autoclaved and the mice received chow and water ad libitum. All animal studies were accepted by the Experimental Animal Committee of the University of Eastern Finland.

### Histology, Immunohistochemistry, Microvessel Measurements and Real-Time Quantitative PCR

Tissue samples were immersed in 4% paraformaldehyde for 4-6 h, followed by overnight immersion in 15% sucrose.²⁴ The specimens were embedded in paraffin and 5 μm thick sections were processed for hematoxylin-eosin, Ki-67 (DakoCytomation, Glostrup, Denmark), CD-34 (HyCult biotechnology b.v., AA Uden, The Netherlands) and LYVE-1 (ReliaTech GmbH, Braunschweig, Germany) stainings.

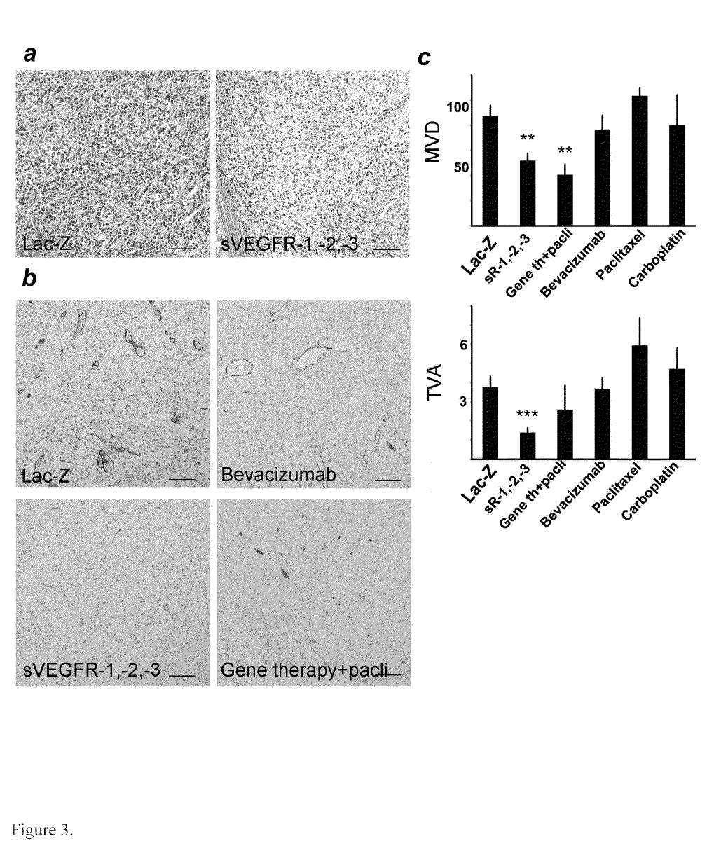

Photographs of histological sections were taken and processed using an Olympus AX70 microscope (Olympus Optical, Japan), and analySIS (Soft Imaging System, GmbH, Germany) and PhotoShop (Adobe) softwares. Microvessel density (MVD) and total microvascular area (%) of the tumors (TVA) were measured from CD34-immunostained sections using analySIS software at 100× magnification in a blinded manner (FIG. 3b). Six to ten different fields which represented maximum microvessel density areas were selected from each tumor. Necrotic areas were avoided. In addition, the total number of LYVE-1 positive lymphatic vessels per section was counted. Means±SEM of the measurements are reported.

Gene expression levels of human and mouse VEGF-A, VEGF-B, VEGF-C, VEGF-D and PLGF in SKOV-3m cells and tumors from AdLacZ-injected mice were determined with real-time quantitative PCR (StepOnePlus instrument and software, Applied Biosystems) with gene expression assays (TaqMan-chemistry based probes, Applied Biosystems).

### Magnetic Resonance Imaging

To follow the development of ovarian carcinoma and to measure tumor volumes, MRI was performed using a 9.4 T vertical magnet (Oxford Instruments, Oxford, UK) equipped with actively shielded field gradients (Magnex Scientific Ltd., Abdington, UK) interfaced to a Varian DirectDrive console (Varian Inc., Palo Alto, Calif., USA).

Mice were anesthetized with an s.c. injection of a mixture of fentanyl-fluanisone (Jansen Pharmaceutica, Hypnorm, Buckinghamshire, UK) and midazolame (Roche, Dormicum 5 mg/ml, Espoo, Finland). For signal transmission and reception, a mouse body surface coil (m2m Imaging Corp., Cleveland, Ohio, USA) was used. Axial T₂-weighted images were acquired (repetition time=2.5 s, echo time=11 ms, field of view=35×35 mm², resolution=256×128, slice thickness=1 mm, number of slices=25). Tumor volumes were measured manually (MatLab, Math-Works Inc., Natick, Mass., USA). The tumor masses differed from surrounding non-tumor soft tissue with intensity and location. To measure tumor volume (mm³), area of the tumor (mm²) was calculated from each slide and then multiplied with summation of the areas by the slice thickness.¹³ If more than one tumor nodule was detected from the MRI scan, the tumor volume was taken as a sum of all nodules. MRI was performed weekly after the first tumors were detectable.

### Clinical Chemistry

Plasma samples were collected at 6, 13, 20 and 27 days after the gene transfer and when the mice were sacrificed. Alanine aminotransferase (ALT) and creatinine (Crea) were monitored using routine clinical chemistry assays at Kuopio University Hospital Central Laboratory. Enzyme-linked immunosorbent assays (Quantikine; R&D Systems, Minneapolis, Minn.) were used to detect the presence of human sVEGFRs in plasma samples.

### Statistical Analyses

Statistical significance was evaluated using Kruskall-Wallis test, followed by Mann-Whitney U-test with correction for multiple comparisons. Kaplan-Meier plots and log rank test were used for the analysis of survival. Results are expressed as mean±SEM. A value of p<0.05 was considered as statistically significant.

### Results

### Transgene Expression

Plasma sVEGFR-1, sVEGR-2 and sVEGFR-3 levels were detectable at all time points by enzyme-linked immunosorbent assay (FIG. 1 in supplementary material). The levels were highest at day 6 after the gene therapy. Plasma level of sVEGFR-1 was minimum of 30.5 ng/ml at day 27 after the treatment where as plasma level of sVEGFR-2 was over 3106.7 ng/ml throughout the follow-up. Plasma level of sVEGFR-3 was 18.1 ng/ml at the time of sacrifice. In the control group, no signals were detected for the soluble receptors at any time point. The plasma levels of sVEGFR-1, -2 and -3 behaved the same way as in our earlier study with soluble receptors.¹² Reverse transcription-PCR with 35 cycles has confirmed mRNA expression of all trangenes in liver samples 6 days after the gene transfer (data not shown).¹²

### Intraperitoneal Tumor Growth

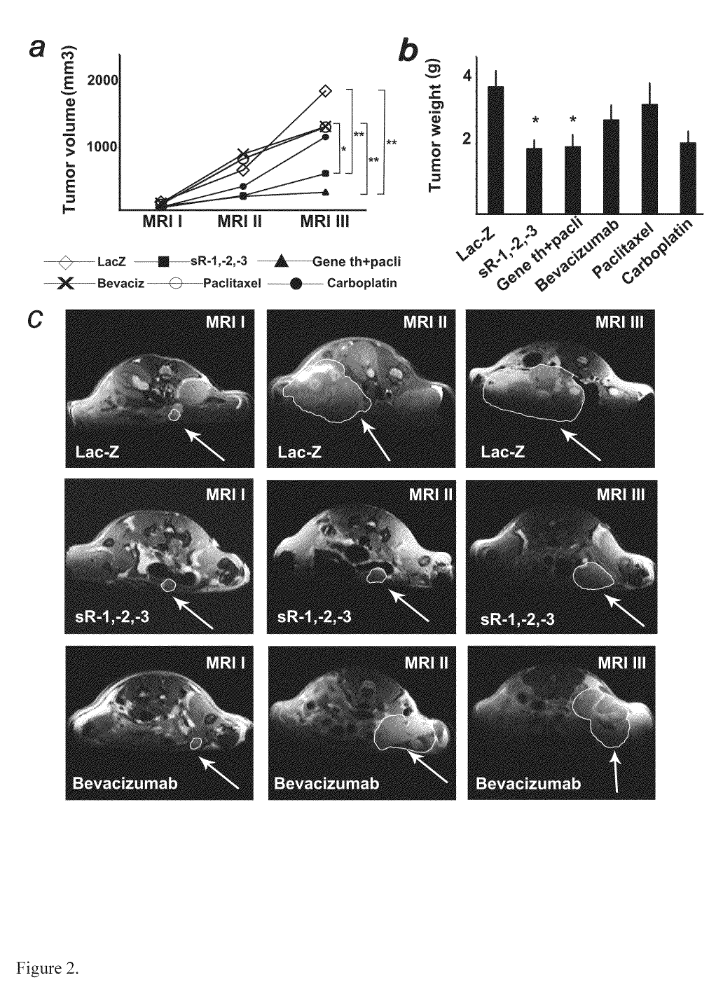

All mice developed intraperitoneal tumors within three weeks (6-21 days) after SKOV-3m cell inoculation. 62% of the mice were treated within ten days and 35% in 11 to 14 days after the inoculation of the SKOV-3m cells. At the baseline tumor volumes detected by MRI did not differ among the controls and different treatment groups. MRI was repeated weekly after the treatment. In the second MRI (after one week of the treatment) the mean tumor volumes of mice treated by gene therapy and paclitaxel were significantly smaller compared to control mice or mice treated with bevacizumab or paclitaxel (p=0.001). Tumor volumes in the gene therapy group were also significantly smaller than in the control (p=0.014), bevacizumab (p=0.0005) and paclitaxel groups (p=0.006) (FIG. 2a,c).

At the time of the third MRI (two weeks after the treatment) the mean tumor volume in the combined gene therapy and paclitaxel group was 85% smaller (p=0.002) than in the control, 79% smaller than in bevacizumab (p=0.006) and paclitaxel (p=0.006) groups and 76% smaller than in carboplatin group (p=0.046). At the same time point, tumor volumes were also significantly smaller in the gene therapy group compared to the control, bevacizumab and paclitaxel groups (p=0.004; p=0.036; p=0.036, respectively) (FIG. 2a,c).

At the end of the follow-up the final mean tumor weights of the mice treated by gene therapy were 50% smaller (p=0.021) than in the controls (1.8 g vs. 3.6 g). Additionally, tumors of the gene therapy group were 42% smaller than in the paclitaxel group (p=0.02). Mice treated by gene therapy and paclitaxel had significantly smaller tumors than the controls and the paclitaxel alone (p=0.037, p=0.048, respectively) (Table 1, FIG. 2b).

TABLE 1
Characterization, MRI-volumes and tumor weights of the
study groups at the end of the follow-up (mean ± SEM)

| | Adenoviral vector/Treatment | | | | | |
|---|---|---|---|---|---|---|
| | | | sVEGFR-1 | | | |
| | | sVEGFR-1 | sVEGFR-2 | | | |
| | | sVEGFR-2 | sVEGFR-3, | | | |
| | LacZ | sVEGFR-3 | Paclitaxel | Bevacizumab | Paclitaxel | Carboplatin |
| n | 21 | 21 | 9 | 14 | 9 | 5 |
| MRI volume | 1915.7 ± 380 | 587.1 ± 214 | 268.3 ± 65 | 1342.1 ± 184 | 1333.5 ± 356 | 1174.6 ± 279 |
| (mean, mm³) | | | | | | |
| Tumor weight | 3.6 ± 0.4 | 1.8 ± 0.2 | 1.9 ± 0.3 | 2.7 ± 0.4 | 3.1 ± 0.6 | 2.0 ± 0.3 |
| (mean, g) | | | | | | |

In this animal model of ovarian cancer, tumors were poorly differentiated (grade 3) serous cystadenocarcinomas with variable size of nucleus and limited stroma. In mice treated by gene therapy tumor tissue was partly replaced by connective tissue and morphology of the tumors was disturbed (FIG. 3a). The cell proliferation index measured by Ki-67 staining did not show any difference according to the treatment arm nor was there any significant difference in the amount of ascites formation. Lymphatic vessel density measured by the LYVE-staining did not differ significantly between the treatment groups, although it tended to be less intense in the gene therapy group (data not shown). In control tumors both human and mouse VEGF-A and PLGF were the most expressed VEGF types. VEGF-D expression was not detectable in cells or tumors (data not shown).

### Microvessel Measurements

To detect the effect of the treatment on intratumoral microvessels, microvessel density (MVD) and tumor vascular area (TVA) were measured. Both MVD (51.59±6.15) and TVA (1.37±0.25%) of the tumors in the gene therapy group were significantly smaller than in the control (87.04±8.66; p=0.001 and 3.74±0.57%; p=0.0005) and paclitaxel groups. Compared to controls and the paclitaxel group, significantly lower MVD of the tumors in the gene therapy combined with chemotherapy was also observed (p=0.008; p=0.02, respectively). However, bevacizumab did not have any effect on angiogenesis by microvessel measurements (FIG. 3b,c). TVA of the tumors in the gene therapy group was also significantly smaller than in the bevacizumab and carboplatin groups (p=0.005; p=0.002, respectively) (FIG. 3c).

### Survival and Safety

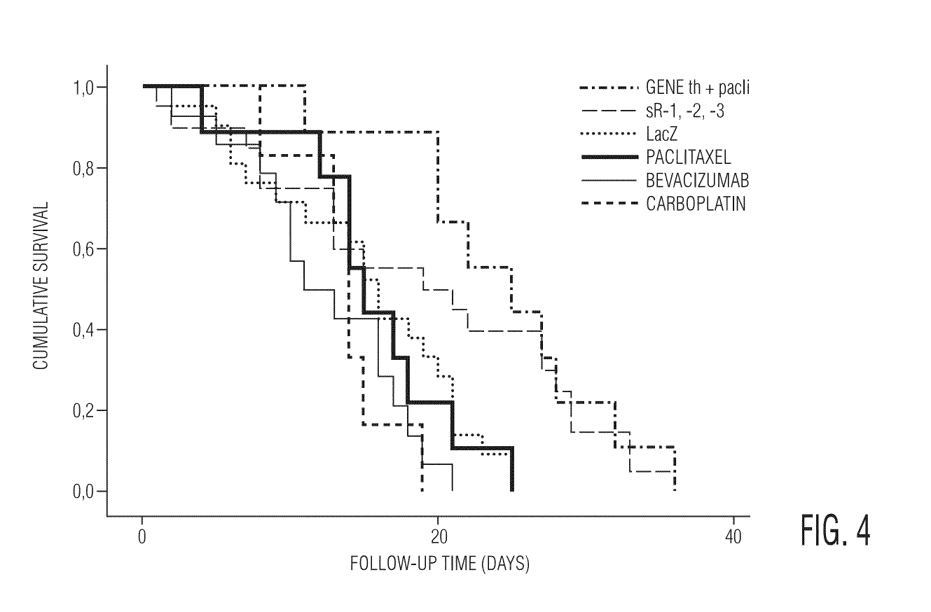

The mean survival (days) was significantly longer in the combined gene and chemotherapy group (25±2 days) compared to the control group (15±1 days) and the other treatment groups (p=0.001). The mean survival of the gene therapy group (19±2 days) was also significantly prolonged compared to the bevacizumab group (p=0.05). On the contrary, the mean survival of the bevacizumab group (12±1 days) did not differ significantly from the survival of the control group. Overall, gene therapy with paclitaxel prolonged the survival of mice compared to all other treatment groups (p=0.001). The mean survival times of the other treatment groups were as follows: 15±1 days in paclitaxel and 13±1 days in carboplatin group (FIG. 4).

Safety was judged by the assessment of the histological samples of liver, spleen, kidneys and lungs as well as by the analysis of plasma ALT and Crea levels. Liver samples of both treated and control mice were normal six days after the gene transfer. At the end of the follow up there was evidence of regenerative changes in the control and gene therapy treated groups, which consisted of stronger atypical changes, large variation in shape and size of the nucleoli and local necrosis especially in the combination gene and chemotherapy group (data not shown). Plasma ALT levels were elevated at the end of the follow-up time in both treatment and control groups. By that time there were wide metastatic changes in the liver. In most of the treatment groups the raise of the ALT levels seemed to be transient with the highest level 13 days after the treatment. Overall, ALT rise was associated with gene therapy and no significant difference in ALT levels could be observed between LacZ, sVEGFR or sVEGFR together with the paclitaxel treatment arms. Creatinine values were within normal range (Table 2).

TABLE 2
Clinical chemistry after the treatment (mean ± SEM)

| | ALT (U/l) | | | Crea (um/l) | | |
|---|---|---|---|---|---|---|
| Group | Day 6 | Day 13 | Day 20 | Day 6 | Day 13 | Day 20 |
| LacZ | 337.1 ± 107 | 211.2 ± 51 | 80.1 ± 11 | 16.8 ± 4 | 27 ± 7 | 21.4 ± 28 |
| sVEGFR-1,-2,-3 | 143.4 ± 32 | 309.1 ± 74 | 204.1 ± 64 | 15.6 ± 3 | 16.7 ± 4 | 18.1 ± 7 |
| sVEGFR-1,-2,-3 + | 67.4 ± 16 | 184.2 ± 67 | 81.3 ± 72 | 9.4 ± 1 | 15.0 ± 5 | 6.8 ± 2 |
| Paclitaxel | | | | | | |
| Bevacizumab | 12.9 ± 3 | 56.3 ± 22 | | 7.4 ± 1 | 12.4 ± 4 | |
| Paclitaxel | 11.3 ± 2 | 83.0 ± 75 | 69.1 ± 23 | 6.6 ± 2 | 5.7 ± 1 | 6.1 ± 3 |
| Carboplatin | 10.0 ± 1 | 399 ± 209 | | 14.4 ± 3 | 4.9 ± 2 | |

Neither was there macroscopic nor microscopic alterations observed in the other organs (data not shown).

### Discussion

In the present study, we demonstrate a survival advantage of antiangiogenic sVEGFR gene therapy together with paclitaxel chemotherapy. Control arms included chemotherapy regimens which are included in the standard treatment of epithelial ovarian cancer as well as bevacizumab, the VEGF-inhibiting antibody. In a mouse model of macroscopic ovarian cancer detected by MRI imaging, the mean survival was significantly better in mice treated by gene therapy combined with chemotherapy compared to the control and other treatment groups, the mean survival advantage being 67%. Gene therapy was also more efficient than the anti-VEGF-antibody in the treatment of ovarian cancer.

The antiangiogenic effect of VEGF receptors 1, 2 and 3 is due to a decoy effect of the soluble receptors. VEGFR-1, -2 and -3 have high binding activity towards VEGFs, but they have no signal transduction domains.²⁵ They act as VEGF antagonists by competing with the native VEGF receptors and inhibit angiogenesis and new vessel formation, which is vital for the tumor growth and metastasis. This VEGF inhibitory effect was observed by the lower microvessel density as well as smaller vascular area of the tumors. Additionally, nutritional depletion was noticed as a diminished tumor volume and weight by sVEGFR gene therapy.

The standard first-line cytotoxic chemotherapy of ovarian cancer consists of paclitaxel and platin-based compounds, mainly carboplatin. Studies have concluded that both cisplatin and paclitaxel arrest the cell cycle at G1 or G2/M followed by double-stranded DNA brakes consistent with apoptosis²⁶. The additional effect of VEGF-targeted therapy on the cytotoxic chemotherapy has been hypothesized to be a result of vessel normalization after VEGF inhibition²⁷ although multiple other mechanisms may exist. In this animal model we could demonstrate a prolonging effect on survival by adding chemotherapy to sVEGFR treatment. The mean survival was 32% longer in the gene therapy combined with chemotherapy arm than in the gene therapy alone. Our results suggest that gene therapy can be added to the chemotherapy without any major toxicity also in ovarian cancer.

The monoclonal anti-VEGF-antibody, bevacizumab, dosed two times per week, did not have any significant effect on survival, tumor volume or mean tumor weight. Bevacizumab is most effective against human VEGF, but it reacts less well against mouse VEGF.²⁸ However, because SKOV-3m cell line is from human origin, bevacizumab should neutralize VEGF produced by these cells. Mouse-derived VEGF-A secreted from bone-marrow derived cells, fibroblasts and other cells in tumor microenvironment contributes to the progression of carcinogenesis. Therefore, it could be possible that the efficacy of this antibody was underestimated in the present study. It may be possible that dosing by intraperitoneal route in this aggressive model with fast growing tumors is less effective than systemic VEGF targeting. Another reason for poor treatment effect may be that in our study the mice had macroscopic tumors at the time of treatment compared to microscopic phase of the tumor progression in earlier studies.^{29,30} In addition, we did not observe any significant difference in ascites formation. However, the dose as well as dosing schedule of bevacizumab was similar in this work compared to others, where positive effects have been observed.³¹

In human studies, bevacizumab as a single-agent has shown comparable activity to chemotherapeutic single agents in recurrent ovarian cancer.^{32,33} In the clinical phase bevacizumab is mainly used for consolidation and maintenance treatment as well as treatment for ascites. Soluble VEGF decoy receptor (VEGF Trap) combined with paclitaxel has prolonged the survival and has shown its antiangiogenic influence on tumor growth and ascites formation.³⁴ In that study the mice were treated intraperitoneally two weeks after the inoculation of the tumor cells without confirming whether there was a visible tumor or not. Combination treatment had also an influence on tumor metastasis and induction of cell apoptosis. Other studies with soluble VEGFR-1/Flt-1 have also shown efficacy on tumor growth and ascites formation.^{25,35} Tumor cells were injected both subcutaneously and intraperitoneally and there was no survival benefit in the ip. group. Several small molecule tyrosine kinase inhibitors targeting VEGFRs have been investigated in phase II studies in relapsed ovarian cancer with some response, but the duration of the effect has been limited and the continuous dosing of the regimen is being explored.³⁶

Liver toxicity has been reported previously when adenoviral sVEGFR-1 has been used intravenously.³⁷ According to our earlier study the histology of the liver samples was normal at the time of the highest sVEGFR-1 levels.¹² However, there were regenerative changes in the liver samples of the mice treated by gene therapy at the end of the follow-up. These findings may also be contributed by severe, widely metastasized intraperitoneal carcinosis. In this study we used the maximum doses of adenoviral sVEGFRs, although lower levels of soluble receptors might reduce liver toxicity without compromising the treatment effect. Liver enzyme levels may also reflect the very aggressive behavior of the SKOV-3m cell line, the first visible tumors arising usually <10 days after inoculation of the cells. The highest ALT levels were measured 13 days after the treatment and before sacrifice in all study groups (Table 2).

This cancer xenograft model resembles the clinical setting of human ovarian cancer with wide intra-abdominal metastasis. The diagnostic and regular monitoring of the tumors in mice by MRI and gene therapy dosed intravenously makes this model also more challenging to establish. In conclusion, we show a survival benefit of up to 67% after antiangiogenic gene therapy with the combination of sVEGFR-1, -2 and -3 and paclitaxel compared to the controls, those receiving the antiangiogenic treatment with the monoclonal anti-VEGF-antibody bevacizumab or single chemotherapy. Potential of the antiangiogenic gene therapy with sVEGFR-1, -2 and -3 as a treatment against human ovarian carcinoma clearly warrants further studies and finally testing in a clinical setting.

Given our disclosure, the skilled artisan will readily be able to devise variants of our disclosure here. For example, one could use docetaxel or another analogous chemotherapeutic agent rather than the paclitaxel we used in our experiments. Similarly, one could use a native VEGF receptor, or a variant engineered to be less immunogenic, or more stable etc. Thus, we intend the ambit of our patent to be defined not by the specific examples we discuss here, but by the legal claims appended here and the legal equivalents thereof.

### INFORMATION DISCLOSURE STATEMENT

1. Jemal A., Siegel R., Xu J, Ward E. Cancer statistics, 2010. CA Cancer J Clin 2010; 60:277-300.
2. Ozols R F, Bundy B N, Greer B E, Fowler J M, Clarke-Pearson D, Burger R A, Mannel R S, DeGeest K, Hartenbach E M, Baergen R. Phase III trial of carboplatin and paclitaxel in patients with optimally resected stage III ovarian cancer: A Gynecologic Oncology Group study. J Clin Oncol 2003; 21:3194-200.
3. Folkman J. Angiogenesis in cancer, vascular, rheumatoid and other disease. Nat Med 1995; 1:27-31.
4. Yamamoto S, Konishi I, Mandai M, Kuroda H, Komatsu T, Nanbu K, Sakahara H, Mori T., Expression of vascular endothelial growth factor (VEGF) in epithelial ovarian neoplasms: correlation of clinicopathology et patient survival and analysis of serum VEGF levels. Br J Cancer 1997; 76:1221-27.
5. Sowter H M, Corps A M, Evans A L, Clark D E, Charnock-Jones D S, Smith S K. Expression and localisation of the vascular endothelial growth factor family in ovarian epithelial tumors. Lab Invest 1997; 77:607-14.
6. Olsson A K, Dimberg A, Kreuger J, Claesson-Welsh L. VEGF-receptor signalling—in control of vascular function. Nat Rev Mol Cell Biol 2006; 7:359-71.
7. Shibuya M. Differential roles of vascular endothelial growth factor receptor-1 and receptor-2 in angiogenesis. J Biochem Mol Biol 2006; 39:469-78.
8. Yokohama Y, Charnock-Jones D S, Licence D, Yanaihara D, Hastings J M, Holland C M, Emoto M, Umemoto M, Sakamoto T, Sato S, Mizunuma H, Smith S K. Vascular endothelial growth factor-D is an independent prognostic factor in epithelial ovarian cancer. Br J Cancer 2003; 88:237-44.
9. Nishida N, Yano H, Komai K, Nishida T, Kamura T, Kojiro M. Vascular endothelial growth factor C and vascular endothelial growth factor receptor 2 are related closely to the prognosis of the patients with ovarian carsinoma. Cancer 2004; 101:1364-74.
10. Monk B J, Han E, Josephs-Cowan C A, Pugmire G, Burger R A. Salvage bevacizumab (rhu MAB VEGF) based therapy after multible prior cytotoxic regimens in advanced refractory epithelial ovarian cancer. Gynecol Oncol 2006; 102:140-4.
11. Raki M, Rein D T, Kanerva A, Hemminki A. Gene Transfer Approaches for Gynecological Diseases. Mol Ther 2006; 14:154-63.
12. Sallinen H, Anttila M, Narvainen J, Koponen J, Hamalainen K, Kholova I, Heikura T, Toivanen P, Kosma V M, Heinonen S, Alitalo K, Yla-Herttuala S. Antiangiogenic gene therapy with soluble VEGFR-1, -2 and -3 reduces the growth of solid human ovarian carsinoma in mice. Mol Ther 2009; 17:278-84.
13. Sallinen H, Anttila M, Narvainen J, Orden M R, Ropponen K, Kosma V M, Heinonen S, Yla-Herttuala S. A highly reproducible xenocraft model for human ovarian carsinoma and application of MRI and ultrasound in longitudinal follow-up. Gynecol Oncol 2006; 103:315-20.
14. Lin X, Zhang Q, Rice J R, Stewart D R, Nowotnik D P, Howell S B. Improved targeting of platinum chemotherapeutics. the antitumour activity of the HPMA copolymer platinum agent AP5280 in murine tumour models. Eur J Cancer 2004; 40:291-7.
15. Yamashita M, Kajiyama H, Terauchi M, Shibata K, Ino K, Nawa A, Mizutani S, Kikkawa F. Involvement of aminopeptidase N in enhanced chemosensitivity to paclitaxel in ovarian carcinoma in vitro and in vivo. Int J Cancer 2007; 120:2243-50.
16. Gerber H P, Ferrara N. Pharmacology and pharmacodynamics of bevacizumab as monotherapy or in combination with cytotoxic therapy in preclinical studies. Cancer Res 2005; 65:671-80.
17. Bhardwaj S, Roy H, Karpanen T, He Y, Jauhiainen S, Hedman M, Alitalo K, Yla-Herttuala S. Periadventitial angiopoietin-1 gene transfer induces angiogenesis in rabbit carotid arteries. Gene Ther 2005; 12:388-94.
18. Pajusola K, Aprelikova O, Armstrong E, Morris S, Alitalo K. Two human FLT4 receptor tyrosine kinase isoforms with distinct carboxy terminal tails are produced by alternative processing of primary transcripts. Oncogene 1993; 8:2931-7.
19. Takayama K, Ueno H, Nakanishi Y, Sakamoto T, Inoue K, Shimizu K, Oohashi H, Hara N. Suppression of tumor angiogenesis and growth by gene transfer of a soluble form of vascular endothelial growth factor receptor into a remote organ. Cancer Res 2000; 60:2169-77.
20. Makinen T, Jussila L, Veikkola T, Karpanen T, Kettunen M I, Pulkkanen K J, Kauppinen R, Jackson D G, Kubo H, Nishikawa S, Yla-Herttuala S, Alitalo K. Inhibition of lymphangiogenesis with resulting lymphedema in transgenic mice expressing soluble VEGF receptor-3. Nat Med 2001; 7:199-205.
21. Veikkola T, Jussila L, Makinen T, Karpanen T, Jeltsch M, Petrova T V, Kubo H, Thurston G, McDonald D M, Achen M G, Stacker S A, Alitalo K. Signalling via vascular endothelial growth factor receptor-3 is sufficient for lymphangiogenesis in transgenic mice. EMBO J. 2001; 20:1223-31.
22. Laitinen M, Pakkanen T, Donetti E, Baetta R, Luoma J, Lehtolainen P, Viita H, Agrawal R, Miyanohara A, Friedmann T, Risau W, Martin J F, et al. Gene transfer into the carotid artery using an adventitial collar: comparison of the effectiveness of the plasmid-liposome complexes, retroviruses, pseudotyped retroviruses, and adenoviruses. Hum Gene Ther 1997; 8:1645-50.
23. Puumalainen A M, Vapalahti M, Agrawal R S, Kossila M, Laukkanen J, Lehtolainen P, Viita H, Paljarvi L, Vanninen R, Yla-Herttuala S. Beta-galactosidase gene transfer to human malignant glioma in vivo using replication-deficient retroviruses and adenoviruses. Hum Gene Ther 1998; 9:1769-74.
24. Yla-Herttuala S, Rosenfeld M E, Parthasarathy S, Glass C K, Sigal E, Witztum J L, Steinberg D. Colocalization of 15-lipoxygenase mRNA and protein with epitopes of oxidized low density lipoprotein in macrophage-rich areas of atherosclerotic lesions. Proc Natl Acad Sci USA 1990; 87:6959-63.
25. Takei Y, Mizukami H, Saga Y, Yoshimura I, Hasumi Y, Takayama T, Kohno T, Matsushita T, Okada T, Kume A, Suzuki M, Ozava K. Suppression of ovarian cancer by muscle-mediated expression of soluble VEGFR-1/Flt-1 using adeno-associated virus serotype 1-derived vector. Int J Cancer 2007; 120:278-84.
26. Gibb R K, Taylor D D, Wan T, O'Connor D M, Doering D L, Gercel-Taylor C. Apoptosis as a measure of chemosensitivity to cisplatin and taxol therapy in ovarian cancer cell lines. Gynecol Oncol 1997; 65:13-22.
27. Ellis L M, Hicklin D J. VEGF-targeted therapy: mechanisms of anti-tumour activity. Nat Rev Cancer 2008; 8:579-91.
28. Presta L G, Chen H, O'Connor S J, Chisholm V, Meng Y G, Krummen L, Winkler M, Ferrara N. Humanization of an anti-vascular endothelial growth factor monoclonal antibody for the therapy of solid tumors and other disorders. Cancer research 1997; 57:4593-99.
29. Mesiano S, Ferrara N, Jaffe R B. Role of vascular endothelial growth factor in ovarian cancer: Inhibition of ascites formation by immunoneutralization. Am J Pathol 1998; 153:1249-56.
30. Limin Hu, Hofmann J, Zaloudek C, Ferrara N, Hamilton T, Jaffe R B. Vascular endothelial growth factor immunoneutralization plus paclitaxel markedly reduces tumor burden and ascites in athymic mouse model of ovarian cancer. Am J Pathol 2002; 161:1917-24.
31. Mabuchi S, Terai Y, Morishige K, Tanabe-Kimura A, Sasaki H, Kanemura M, Tsunetoh S, Tanaka Y, Sakata M, Burger R A, Kimura T, Ohmichi M. Maintenance treatment with bevacizumab prolongs survival in an in vivo ovarian cancer model. Clin Cancer Res 2008; 14:7781-9.
32. Burger R A, Sill M W, Monk B J, Greer B E, Sorosky J I. Phase II trial of bevacizumab in persistent or recurrent epithelial ovarian cancer or primary peritoneal cancer: a Gynecologic Oncology Study. J Clin Oncol 2007; 25:5165-71.
33. Cannistra S A, Matulonis U A, Penson R T, Hambleton J, Dupont J, Mackey H, Douglas J, Burger R A, Armstrong D, Wenham R, McGuire W. Phase II study of bevacizumab in patients with platinum resistant ovarian cancer or peritoneal serous cancer. J Clin Oncol 2007; 25:5180-6.
34. Limin Hu, Hofmann J, Holash J, Yancopoulos G D, Sood A K, Jaffe R B. Vascular endothelial growth factor Trap combined with paclitaxel strikingly inhibits tumor and ascites, prolonging survival in a human ovarian cancer model. Clin Cancer Res 2005; 11:6966-71.
35. Hasumi Y, Mizukami H, Urabe M, Kohno T, Takeuchi K, Kume A, Momoeda M, Yoshikawa H, Tsuruo T, Shibuya M, Taketani Y, Ozawa K. Soluble Flt-1 expression suppresses carsinomatous ascites in nude mice bearing ovarian cancer. Cancer Res 2002; 62:2019-23.
36. Matulonis U A, Berlin S, Ivy P, Tyburski K, Krasner C, Zarwan C, Berkenblit A, Campos S, Horowitz N, Cannistra S A, Lee H, Lee J, et al. Cediranib, an oral inhibitor of vascular endothelial growth factor receptor kinases, is an active drug in epithelial ovarian, fallopian tube and peritoneal cancer. J Clin Oncol 2009; 27:5601-6.
37. Parameshwar J M, Manjula K, Ming H W, Cecil R S, William E G. Intravenous delivery of adenovirus-mediated soluble FLT-1 results in liver toxicity. Clin Cancer Res. 2003; 9:2701-10.

We claim:

1. In a method of treating cancer by administering a cytotoxic compound, the improvement comprising administering a decoy receptor which binds to Vascular Endothelial Growth Factor, said cytotoxic compound and said decoy receptor administered in amounts which are together effective to treat said cancer.

2. The method of claim 1, wherein said decoy receptor comprises at least one compound selected from the group consisting of: Flt-1; KDR; Flt-4; a derivative of Flt-1, KDR or Flt-4; and an analogue of Flt-1, KDR or Flt-4.

3. The method of claim 1, wherein said decoy receptor is in a form adequate to ensure efficient dimerization of said decoy receptor.

4. The method of claim 1, said decoy receptor characterized in being soluble in normal saline.

5. The method of claim 1, wherein said decoy receptor does not initiate VEGF signal transduction.

6. The method of claim 1, wherein said decoy receptor comprises polypeptide, and wherein said administering a decoy receptor comprises administering a gene therapy vector comprising an expressible transgene coding for said decoy receptor polypeptide.

7. The method of claim 6, wherein said cytotoxic compound is administered about one week after said gene therapy vector is administered.

8. The method of claim 1, wherein said cytotoxic compound comprises taxane.

9. The method of claim 1, wherein said cancer comprises cancer of mesothelium tissue.

10. The method of claim 9, wherein said mesothelium tissue comprises ovarian tissue.

11. The method of claim 1, wherein said cytotoxic compound is administered intra-peritoneally and said decoy receptor is administered intravenously.

12. A kit comprising a cytotoxic compound and a decoy receptor which binds to Vascular Endothelial Growth Factor, said cytotoxic compound and said decoy receptor in amounts which are together effective to treat said cancer.

13. The kit of claim 12, wherein said decoy receptor comprises at least one compound selected from the group consisting of: Flt-1; KDR; Flt-4; a derivative of Flt-1, KDR or Flt-4; and an analogue of Flt-1, KDR or Flt-4.

14. The kit of claim 12, wherein said decoy receptor is in a form adequate to ensure efficient dimerization of said decoy receptor.

15. The kit of claim 12, said decoy receptor characterized in being soluble in normal saline.

16. The kit of claim 12, wherein said decoy receptor does not initiate VEGF signal transduction.

17. The kit of claim 12, wherein said decoy receptor comprises polypeptide, and wherein said administering a decoy receptor comprises administering a gene therapy vector comprising an expressible transgene coding for said decoy receptor polypeptide.

18. The kit of claim 17, wherein said cytotoxic compound is administered about one week after said gene therapy vector is administered.

19. The kit of claim 12, wherein said cytotoxic compound comprises taxane.

20. The kit of claim 12, wherein said cancer comprises cancer of mesothelium tissue.

21. The kit of claim 20, wherein said mesothelium tissue comprises ovarian tissue.

22. The kit of claim 12, wherein said cytotoxic compound is administered intra-peritoneally and said decoy receptor is administered intravenously.
